(19) United States
(12) Patent Application Publication
(10) Pub. No.: US 2005/0084951 A1
(43) Pub. Date: Apr. 21, 2005

(54) Microcapillary bioreactor for growth of biological tissues

(76) Inventors: Shidfar J Rouhani, Gilbert, AZ (US); David G. Capco, Tempe, AZ (US)

Correspondence Address: Richard E Oney;Fennemore Craig, Suite 2600, 3003 North Central, Phoenix, AZ, 85012-2913, US

(21) Appl. No.: 10/505,911

(22) PCT Filed: Feb. 28, 2003
(86) PCT No.: PCT/US03/06251

Related U.S. Application Data
(60) Provisional application No. 60/360,918, filed on Feb. 28, 2002.

(51) Int. Cl. C12M 1/34

(52) U.S. Cl. 435/287.1

(57) ABSTRACT

The invention provides an apparatus and method to grow tissues in a three-dimensional mass, in a fashion similar to the way tissues grow in the mammalian body. A bioreactor according to the invention permits the growth and/or maintenance of biological tissue having a normal histotype, unlike prior art or methodology. The bioreactor further permits viewing of the tissues without disturbing the tissues.

### RELATED APPLICATION DATA

This application is based on and claims the benefit of U.S. Provisional Patent Application No. 60/360,918 filed on Feb. 28, 2002.

### FIELD OF THE INVENTION

This invention relates to a device and method for culturing and studying cells, tissues and organs.

The study of cells and tissues has long been performed via the static growth of tissue and cells in a petri dish or plate. However, static cultures lack many of the in vivo intricacies that effect the growth, maturation, and sustenance of living tissue. Therefore, tissues and cells grown in petri dishes do not tend to exhibit identical characteristics to those found in a living body. In a dynamic system, as is normally found in living systems, nutrients and vital gases are constantly delivered while harmful toxins and waste are continually removed.

In contrast, the static environment of a petri dish depends largely on the diffusion of nutrients and waste. The pitfall of a diffusion dependent environment is that a significant concentration gradient must exist in order to drive the diffusion Process. In the absence of a concentration gradient, the tissue is nutritionally starved and surrounded by potentially toxic waste, until there is a significant-concentration difference for the macromolecules to move. Tissue grown in petri dish is also forced to conform to a flat two-dimensional environment, in contrast to the three-dimensional environment from which it originated.

### DESCRIPTION OF THE RELATED ART

There have been a number of approaches for growing tissue/cells in three-dimensional mass. The factors that limit the growth of cells in a three-dimensional mass include the ability to obtain nutrients and oxygen, and the ability to remove waste and carbon dioxide. In mammalian bodies, including human bodies, the circulatory system normally provides these exchanges (see Tortora, Gerard J., et al., Principles of Anatomy and Physiology, 7^{th} Edition, Harper Collins Publishing, 1992; incorporated herein by reference). Examples of prior approaches range from the relatively simple approach of using a dish referred to as an organ-culture dish (see Freshney, R. I., et al., Culture of Animal Cells: A Manual of Basic Technique, 3rd Edition, Wiley-Liss Inc., 1994; incorporated herein by reference), to more complex systems such as making a two-dimensional slice through an organ (see Harris and Stewart, “Propagation of Synchronous Epileptiform Events From Subiculum Backward Into Area CA1 of Rat Brain Slices”, Brain Res. 895, pp. 41-49, 2001; Victorov, I., et al., “A Modified Roller Bottle Method for Organotypic Brain Cultures: Free-Floating Slices of Postnatal Rat Hippocampus”, Brain Res. Protocols 7, pp. 30-37,2001; both incorporated herein by reference). In the organ culture dish, small chunks of tissue are collected into the center of the dish by a conically sloped surface. While the cells in aggregates or chunks placed in such a dish retain their histotype for longer time intervals than when placed directly onto the surface of the traditional cell culture plate, the organization of the tissue is disrupted over time.

In order to circumvent the aforementioned disruption of tissue, some investigators have taken the approach of using slices of tissue from an organ that retain tissue organization best in two-dimensions, that is length and width, while the depth of the tissue slice is limited to only a few cell layers to permit good nutrient/waste/gas exchange. This latter approach is exemplified by experiments with brain slices. However, brain slices that are 300-450 micrometers thick are limited in viability by the amount of oxygen reaching cells in the center of the tissue slice, although the viability of such tissue is increased when the culture is incubated in hyperbaric oxygen (see Mulkey, et al., “Oxygen Measurements in Brain Stem Slices Exposed to Normobaric Hyperoxia and Hyperbaric Oxygen”, J. App. Physiol. 90, pp. 1887-1899, 2001, incorporated herein by reference).

U.S. Pat. No. 6,001,585 discloses a hollow fiber bioreactor of the prior art. Prior technology provides a means of using bioreactors for generating biological compounds, harvesting cells, producing biopharmaceutical by cells, and expanding cell populations rapidly. The bioreactor chamber of the prior art provides a growth surface for cells, which multiplies with the number of fibers potted in the chamber. However, inoculation in previous bioreactor designs requires cells to be trypsinized, pelleted by centrifugation, and resuspended before injection into the extra-capillary space.

The prior art bioreactors are also not capable of allowing the placement and removal of large aggregates of cells (neither tissues nor organs) from among the fibers because there is no means of inserting the three-dimensional mass of tissue. One of the features of the present invention is an access door allowing the placement and removal of tissue in any desired orientation from among the fibers. The present invention permits sections of tissue to be inserted into the bioreactor, with minimal or no disruption to the tissue structure organization. In contrast to the limitation of the prior art, the present invention provides a means for the user to grow tissues, open the bioreactor door to biopsy the tissue and close the door to continue the experiment aseptically and without interruptions.

Previous commercially available bioreactors are constructed of plastic cylindrical casings that are too large for proper microscope staging. Moreover, the round shape lacks stability on microscope stages. The present invention, however, features a stable, flat design with optically clear glass viewing windows for optimal photographic quality under a microscope.

Yet another important device feature according to the present invention is a replaceable fiber cartridge design. This enables a single device to be used multiple times by simply changing the microcapillary fiber with a new fiber cartridge with similar or different fiber characteristics. The design's replaceable cartridge, tissue access door, as well as its viewing and photography capabilities are expected to enable future studies to document unobserved biological phenomena in conditions that mimic the in vivo environment.

The present invention overcomes the aforementioned drawbacks of culturing in a petri dish or plate, via a bioreactor for growing three-dimensional aggregates of tissue in a fashion that closely mimics the in vivo environment. The invention provides a novel way to grow tissues in a three-dimensional mass, in a fashion similar to the way tissues grow in the mammalian body. The invention permits the growth and/or maintenance of histotype, unlike prior art methodology. The bioreactor has been shown to permit the successful maintenance of viable mouse ovary explants, the observation of continual oocyte maturation and release, and also provides for the three-dimensional adherence of tissue to a microcapillary scaffold mimicking the circulating system. (This was in stark contrast to control mouse ovary explants observed in a petri dish, which exhibited minimal viability of superficial cells, lacked any sort of oocyte maturation or release, and failed to adhere to the flat two-dimensional surface of the petri dish.)

### BRIEF SUMMARY OF THE INVENTION

The present invention provides a bioreactor that employs semi-permeable microcapillary fibers to replace the blood vessels normally found in tissue, for the purpose of growing cells in an ex vivo matrix/scaffold environment. Specifically, the bioreactor uses microcapillary fibers to continuously carry nutrients (oxygen, glucose, etc.) to the tissue, and to continuously carry away waste products (carbon dioxide, urea, etc.) from the tissue, whilst encouraging the growth of “normal” tissue histotype. The bioreactor of the present invention includes the following components: a chamber for growing tissue of interest, an access door for easy placement of the tissue onto the inner microcapillary fiber scaffolding, a structure which allows for easy viewing of cells while on standard microscope stages, enables circulating medium to pass through the inner capillary fibers, and enables easy syringe injection of fluids, such as hormones, growth factors, pharmaceuticals, toxins, etc., into the chamber's extra-capillary space. The device also allows easy replacement of microcapillary fibers and sterilization of the whole assembly for reuse.

The device is made of material selected for easy sterilization and for bioinertness to cells. Tissues are grown on and amongst the microcapillary fibers, and are able to retain a normal or almost normal three-dimensional structure. Reagents or substances such as hormones, growth factors, pharmaceuticals, toxins, etc. may be introduced into the bioreactor via an extra-capillary input port, and the effects of these substances on viability, tissue morphology, growth patterns and rates, and other biological functions of the tissue can be measured and observed. Those reagents may then be removed from the bioreactor via an extra-capillary output port. The tissues in the bioreactor may be continuously or intermittently viewed such as via a microscope, and pictures or other data recorded. Using the reactor of this invention, the environment, including reagents, to which the tissues are subjected can be closely controlled, and the effect on the tissues closely monitored. The extra-capillary port may also be used to sample the extra-capillary medium, which is analogous to the extra-cellular environment of the body, for changes in medium constituents or cellular metabolical excretions.

The bioreactor's ability to maintain healthy tissue was tested using tissue from the mouse ovary. For these experiments adult ovaries were removed from 7-week-old female mice and sliced into three thick segments. One segment from each ovary was place in the microcapillary bioreactor while another segment from each ovary was placed in a standard cell culture dish and both preparations were covered with cell culture medium. Comparisons were made concerning the health of the thick tissue slices over time. One of the objects of the invention is to provide a means for nutrient/gas exchange in order to promote maintenance of healthy tissue better than the tissue slice in the cell culture dish.

This invention mimics the body's circulatory system environment and allows precise ex vivo control. This invention provides a means for tissue placement and removal access from among microcapillary fibers, microscope observations, assembly sterilization for reuse, and easy replacement of microcapillary fibers.

Problem Definition and Solution.

The purpose of this device is to grow tissues in a three-dimensional mass, in a fashion similar to the way tissues grow in the body. While aggregates of cells placed in an organ-culture dish retain their histotype for longer time intervals than when placed directly onto the surface of the traditional cell culture plate, the organization of the tissue is disrupted over time.

The factors that limit the growth of cells in a three-dimension mass are the ability to obtain nutrients and oxygen in addition to the ability to remove waste and carbon dioxide. The circulatory system normally provides these exchanges (see Tortora, Gerard J., et al., Principles of Anatomy and Physiology, 7^{th} Edition, Harper Collins Publishing, 1992; incorporated herein by reference). The bioreactor of the present invention utilizes semi-permeable microcapillary fibers to replace the blood vessels. This design solution maintains an optimal physiological environment for tissue to remain viable ex vivo and carry on functions that are otherwise difficult or impossible when undergoing static growth in a petri dish.

### DESCRIPTION OF THE DRAWINGS

The accompanying drawings, which are incorporated in and constitute a part of the specification, illustrate the presently preferred embodiments and methods of the invention. Together with the general description given above and the detailed description of the preferred embodiments and methods given below, they serve to explain the principles of the invention.

FIG. 1 is a front perspective view of one embodiment of a bioreactor according to the invention.

FIG. 2 is a rear perspective view of the bioreactor of FIG. 1

FIG. 3 is an exploded view of the bioreactor of FIG. 1.

FIG. 4 is a partial sectional perspective view of the base of the bioreactor of FIG. 1.

FIG. 5 is a front elevation cross-section of the base of the bioreactor of showing the microcapillary fiber cartridge held within the bioreactor with the fibers in a taut position within the chamber.

FIG. 6 is a front elevation cross-section of the base of the bioreactor of showing the microcapillary fiber cartridge held within the bioreactor with the fibers in a loose position within the chamber.

FIG. 7 is a schematic drawing of a bioreactor system utilizing a bioreactor in accordance with the invention.

### DETAILED DESCRIPTION OF THE INVENTION

Reference will now be made in detail to the presently preferred embodiments and methods of the invention as illustrated in the accompanying drawings, in which like reference characters designate like or corresponding parts throughout the drawings.

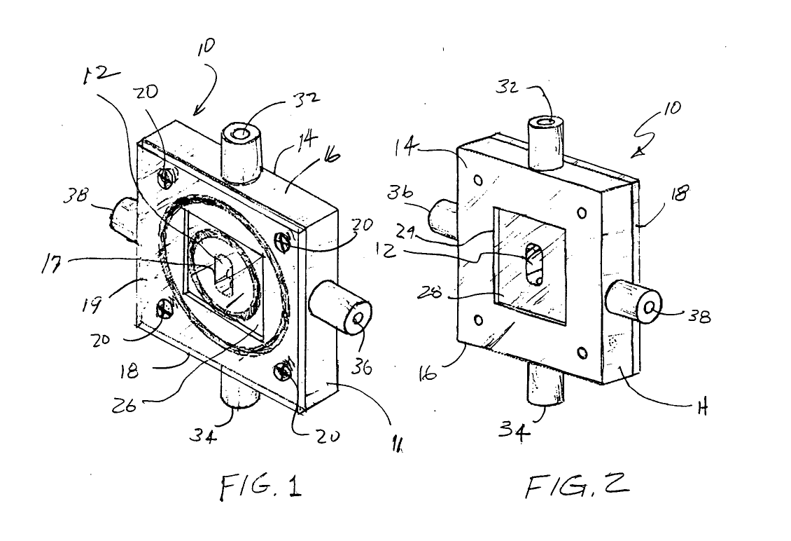

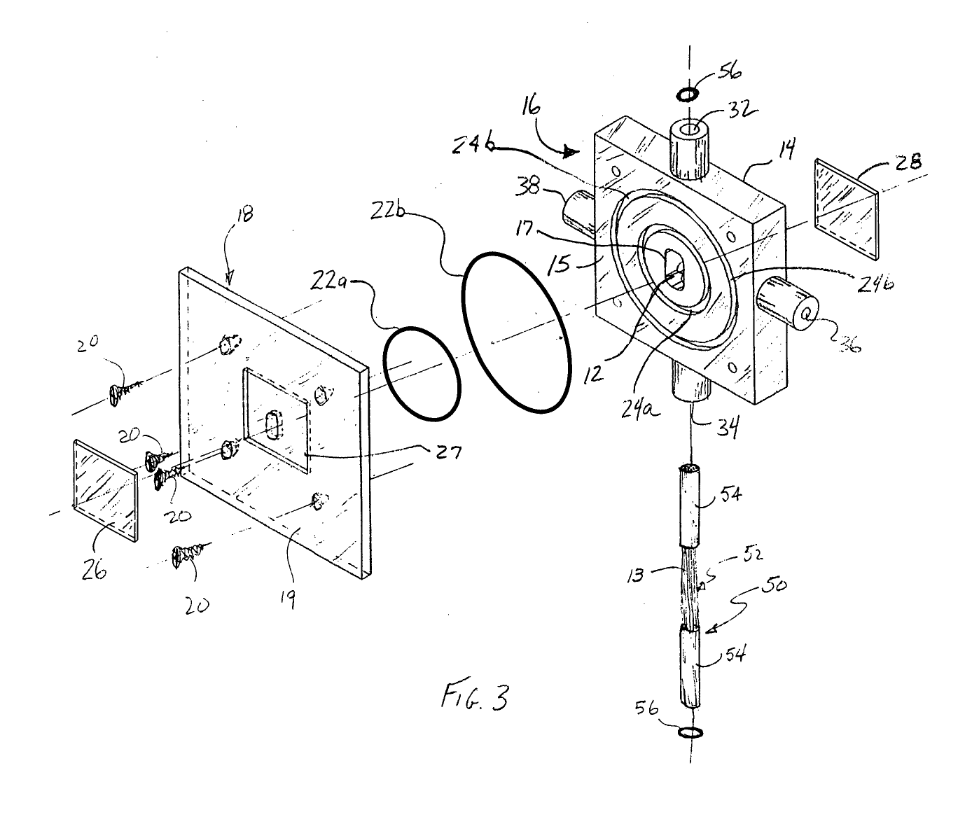

Referring to FIGS. 1 through 6, an illustrative embodiment of a bioreactor 10 according to the present invention is shown. The bioreactor 10 includes a body 11 having a chamber 12 therein for holding the cells or tissue to be grown in the bioreactor. Microcapillary fibers 13 are disposed within and traverse the chamber 12. A circulating medium, typically comprising a nutritive composition, is circulated through the microcapillary fibers 13 (in the intra-capillary space of the fibers, i.e. the space within the bores of the fibers).

The body 11 is generally plate-shaped and includes a base 16 and a cover 18. The base 16 has a generally flat rear face 14 and an opposing, generally flat front face 15. The chamber 12 includes an access opening 17 in the base front face 15. The cover 18 is generally flat and covers the chamber access opening 17 to provide a generally flat front face 19 for the body 11. The cover 18 is removably secured to the base 16 using fasteners 20, such as screws, made of a suitable material. Suitable materials for the fasteners 20 include any material that will not corrode and that will adequately permit fastening of the cover 18 and the base 16. In a preferred embodiment, the fastener is a screw that can be unscrewed to permit the cover 18 and the base 16 to be separated. Preferred fasteners are stainless steel screws. Most preferably the screw heads or other fasteners are flush with or recessed below the cover front face 19. By removing the cover 18 from the base 16, one can load cells or tissue into, or remove cells or tissue from, the chamber 12 of the bioreactor 10.

A seal 22 between the cover 18 and base 16 prevents fluids from leaking into or out of the bioreactor chamber 12 and maintains a sterile environment within the chamber 12. In a preferred embodiment, the seal 22 includes a polymeric O-ring 22a that is seated in a circular groove 24a in the base front face 15. In yet a still more preferred embodiment, the seal 22 also includes a second outer O-ring 22b seated in a second circular groove 24b that is concentric with the inner circular groove 24a; in this configuration, the inner O-ring 22a provides the primary seal, and the outer O-ring 22b provides a secondary seal in the event that the primary seal of the inner O-ring 22a leaks and also prevents ambient air from reaching the inner O-ring 22a, thus keeping it aseptic. The O-rings 22 should be of a polymeric material that is able to withstand autoclaving and be bioinert, and must be sufficiently deformable when the cover 18 and the base 16 are secured together to provide a tight seal.

In this configuration, the bioreactor can be produced to have a thickness that is within the focal length of a microscope so that it can be placed on the stage of a standard microscope to examine the contents of the chamber 12. The bioreactor base 16 and cover 18 are preferably made from a polymeric material having sufficient durability to permit sterilization by autoclaving, ethylene oxide, or other methods. It is preferred that at least a portion of the body 11 of the bioreactor is translucent. In a preferred embodiment, the body 11 is made of a polycarbonate material such as Lexan™, which permits the bioreactor 10 to be autoclaved, and thus reused, about twenty times, or to be sterilized via ethylene oxide and reused indefinitely. In another preferred embodiment, the body is made of acrylic, which cannot be autoclaved but which may be sterilized using ethylene oxide.

The cover 18 includes a viewing window 26 for viewing into the chamber 12 through the cover 18. In addition, the base 16 includes a viewing window 28 for viewing into the chamber 12 through the base 16. In a preferred embodiment both viewing windows 26, 28 are included. It will be understood, however, that either or both of the viewing windows can be included. The windows 26, 28 are preferably of an optically clear material, so as to permit viewing into the chamber 12 using a microscope. It is also preferred that the windows 26, 28 be made of a material which does not lose its visibility due to sterilization procedures. In one advantageous embodiment, the viewing windows 26, 28 are an optically clear glass such as a cover slip for slides. This permits the user to clearly view the contents of the chamber 12 using a microscope, and/or enables clear photographs to be taken of the contents. The front glass viewing window 26 is inserted into a recess 27 in cover 18 and is secured to the cover 18 using an adhesive, such as a medical grade silicone adhesive. Similarly, the rear glass viewing window 28 is inserted into a recess 29 in the base 16 of the bioreactor 10 using such an adhesive. The adhesive must be autoclavable and/or able to withstand ethylene oxide or other chemical sterilization, and be bioinert.

Referring to FIGS. 4-6, a circulating medium passageway 30 extends through the base 16 and has an input port 32 and an output port 34. The circulating medium passageway 30 intersects and is in fluid communication with the chamber 12. Also, an extra-capillary input port 36 is in fluid in communication with the chamber 12 via an extra-capillary input passageway 39, and an extra-capillary output port 38 is in communication with the chamber 12 via an extra-capillary output passageway 40.

As mentioned above, the semi-permeable microcapillary fibers 13 are disposed within the circulating medium passageway 30 and traverse the chamber 12. Each of the of microcapillary fibers 13 has an input end 42 that is in fluid communication with the circulating medium passage input port 32 and an output end 44 in fluid communication with the circulating medium passage output port 34. The interior bores of the microcapillary fibers 13 collectively define an intra-capillary space (not shown). The chamber 12 and the microcapillary fibers 13 define an extra-capillary space 46 that is within the chamber 12 and on the exterior of the fibers 13. The chamber 12, which defines the extra-capillary space 46, can be of virtually any shape or size (volume). In most instances, it is preferable that the volume of the chamber 12 be relatively small, in order to reduce the amount of reagent that need be added to effect the tissue in the chamber. This is often due to cost concerns, because reagents such as hormones and growth factors tend to be quite expensive, and therefore it is desirable to use as small amount of such reagents as is possible. The smaller the volume of the chamber 12, the less reagent that needs to be introduced into the chamber 12 via the extra-capillary input port 36. The larger the portion of tissue desired to be loaded into the bioreactor 10, the larger one will want the chamber 12 to be. The size of the chamber 12 may also be chosen based upon the type of tissue to be grown therein. Thus, if one is growing or maintaining embryos in the bioreactor 10 (for example, in conjunction with human fertility treatments), a bioreactor with a larger chamber 12 may be desirable.

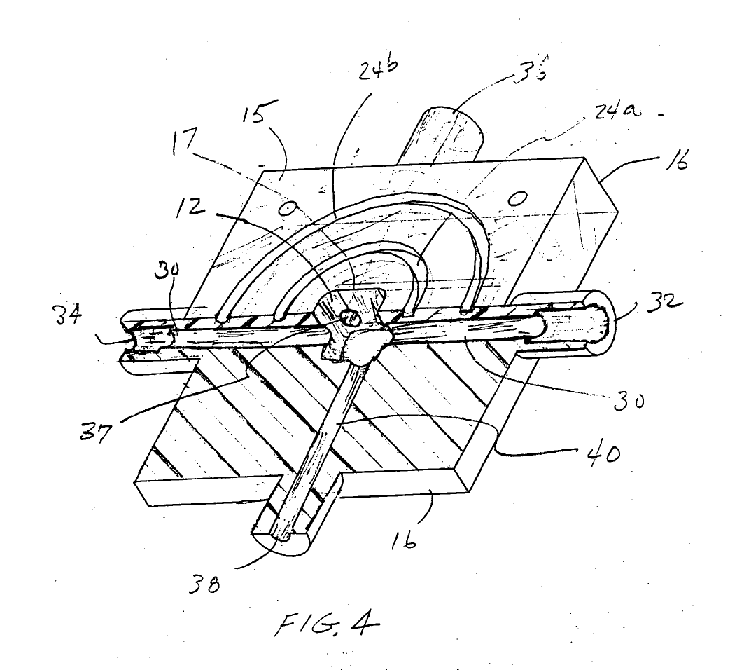

Referring again to FIGS. 3, 5 and 6, in one preferred embodiment, the bioreactor includes a removable microcapillary fiber cartridge 50 that carries the microcapillary fibers 13 in a bundle 52 and is inserted into the circulating medium passageway 30. The cartridge 50 has an end tube 54 disposed over each end of the fiber bundle 52 with ends of the microcapillary fibers 13 extending out of the end tubes 54. The fiber bundle 52 is rigidly held in position within each of the end tubes 54 with a potting material in the end tube 54. In one preferred embodiment, the end tubes 54 are glass tubes and the potting material is a biocompatible silicon adhesive. The end tubes 54 have a diameter that is less than the diameter of the circulating medium passage input port 32 and output port 34, so that the cartridge 50 can be inserted through either of the circulating medium passage input port 32 and output port 34 and positioned with one end tube 54a resting in the circulating medium passage input port 32 and the opposing end tube 54b resting in the circulating medium passage output port 34 with the microcapillary fibers 14 traversing the length of the chamber 12. A polymeric O-ring 56 is disposed around each of the end tubes 54 and is pressed into the circulating medium passage input port 32 and output port 34 so as to provide a seal between the bioreactor chamber 12 and the exterior of the bioreactor 10. In this configuration, the extra-capillary space includes the space within the chamber 12 on the exterior of the fibers 13 as well as the space in the circulating medium passageway 30 that is between the O-rings 56 and the chamber 12 and that is not occupied by the fiber cartridge 50. The seal prevents fluid within the chamber 12 from leaking out, and also prevents circulating fluid from entering the chamber 12. Preferably, the O-rings 56 are composed of a material that is autoclavable and/or able to withstand ethylene oxide or other chemical sterilization, and be bioinert. When it is desired to re-use the bioreactor 10, the microcapillary fiber cartridge 50 can be removed, and a cartridge containing new fibers can be inserted, and the entire assembly is then sterilized. Thus, the cartridge provides a convenient way to re-load the bioreactor 10 for subsequent use.

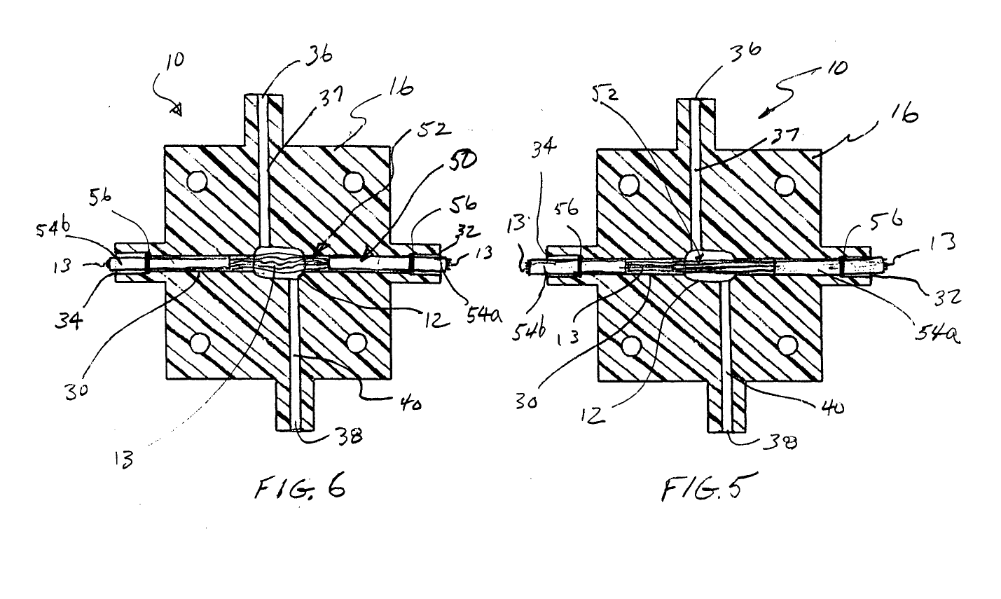

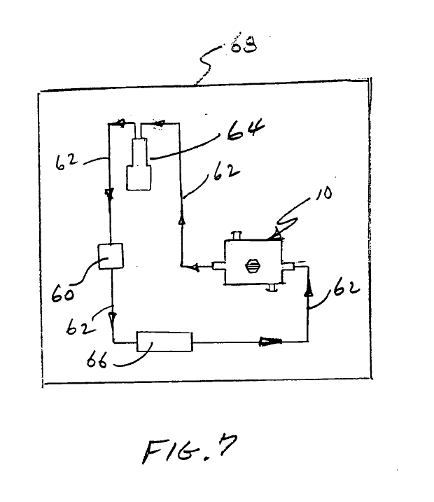

The fiber cartridge 50 also permits the user to adjust the tautness of the microcapillary fibers 13 within the bioreactor 10 by adjusting the position of the end tubes 54 within the circulating medium passage input port 32 and output port 34. Thus, the fibers 13 can be held tautly, as shown in FIG. 5, which is preferred for loose cells, and thus is better for cellular systems not necessarily at the tissue level. Alternatively, the position of the end tubes 54 can be adjusted so that the fibers 13 are held more loosely, as shown in FIG. 6, which permits tissue or pieces of organs to be inserted amongst the fibers 13.

The microcapillary fibers 13 are semi-permeable, permitting the passage through their walls of a range of biological nutrients and waste products. Examples of biological nutrients are glucose and oxygen, although the term biological nutrient is intended to encompass any type of material that assists in sustaining the life of cells and cellular tissue. Examples of biological waste products include carbon dioxide and urea, but this term is intended to encompass any type of byproduct of the biological activities of cells and cellular tissue. Examples of various types of biological nutrients and waste products are set forth in Table 1. The microcapillary fibers 13 bring nutrients to the tissues in the bioreactor 10, and transport out of the tissue waste products, thereby simulating in vivo conditions. This results in the ability to maintain and/or grow tissues in the bioreactor 10 that are identical to or highly similar to in vivo tissues.

In choosing which microcapillary fibers 13 to employ in the bioreactor 10, the user may consider what types and sizes of biological nutrients and waste products are intended to be conveyed into and out of the bioreactor 10. Fibers 13 can then be chosen based on their particular upper and lower ranges of molecular weight permeability. Thus, in most cases it is desired to introduce glucose through the fibers 13, and so fibers having a permeability large enough to permit glucose molecules through must be chosen.

In the case wherein a reagent, such as a hormone, a growth factor, a pharmaceutical, a toxin, etc., is to be added to the bioreactor chamber 12 via the extra-capillary input port 36, yet another consideration in choosing fibers is to choose fibers 13 which are not permeable to the reagent, to avoid the reagent from being carried away via diffusion into the intracapillary circulating medium.

Also with respect to the microcapillary fibers 13, the choice of the material itself of which the fibers are made is important. Depending upon the choice of the material, the fibers 13 can be adherent or nonadherent to cells and tissue. Generally speaking, adherent microcapillary fibers will over the short term tend to slightly disrupt the histotype of the tissue, but will provide the advantage that the tissue will become immobilized on the fibers. An example of an adherent microcapillary fiber is cellulosic fiber, which tends to permit cells to “crawl” or “walk” along the fiber. In contrast, nonadherent fibers will tend not to distort or disrupt the histotype of the tissue, and therefore provide an advantage wherein it is important to retain the histotype of the tissue grown in the bioreactor 10. An example of a nonadherent microcapillary fiber is polysulfone, which tends to not permit cells to “crawl” or “walk” along the fiber, and thus promotes the growth of the tissue in a structure more similar to nature.

Adherent fibers such as cellulosics are thought to be useful wherein one is studying cancer cells within the bioreactor. This is because cancer cells are not anchorage dependent; thus, unlike normal cells which will tend not to go through mitosis unless they are anchored, cancer cells will go through mitosis and proliferate even if they have not adhered to a surface.

Referring to FIG. 7, the bioreactor 10 is used in a system including a pump 60 for circulating media through the microcapillary fibers 13. The circulation of the media through the fibers 13 insures that the cells or tissue within the chamber 12 of the bioreactor 10 are supplied with a constant stream of nutrients, and that waste products are constantly removed, as occurs in vivo. Peristaltic pumps are preferred, because they simulate the natural pulsitile flow of blood through capillaries. However, in certain situations a non-peristaltic pump may be used. For example, non-peristaltic pumps may be used in cases where the cells and/or tissues are sensitive to the pressure change brought about by the peristaltic pump.

The bioreactor system also utilizes tubing 62, preferably silicone tubing, to transport media from a reservoir 64 containing the nutrients to the bioreactor 10, and back to a gas exchange device 66 for removing waste materials from the bioreactor. The reservoir 64 can be easily changed, either to replenish the nutrients or to change the nutrient balance. The gas exchange device 66 can be lengths of silicone tubing wrapped around a stand, which provides large areas for gas exchange, or alternatively can be a radiator-style device in a Teflon bag.

The number of microcapillary fibers 13 to be selected for a particular use in the bioreactor depends in part upon the size of the biological sample to be inserted in to the bioreactor. For example, the larger the piece of tissue, the more microcapillary fibers that will be required to transport nutrients and carry away waste products therefrom. However, the use of too many fibers may obstruct the view of the tissue. Therefore, the user of the bioreactor will have to weigh these factors in determining how many fibers to use in a particular application.

The bioreactor system is placed inside a cell culture incubator 68, wherein atmospheric conditions such as temperature, humidity and gas content are controlled. Typically, the cell culture incubator 68 atmosphere is maintained at about 37 degrees Celsius, at the highest humidity (water saturation) level possible, and contains about 5% carbon dioxide. However, the ratios of gases, humidity levels and temperature can be manipulated as desired. For example, different levels of oxygen may be used, and even hyperbaric oxygen may be used. The bioreactor system utilizes a gas exchange device or mechanism to achieve proper gaseous exchange between the atmosphere in the incubator and the bioreactor. It is preferred to use a gas permeable tubing, more preferably silicone tubing, to transport the circulating media from the reservoir containing the nutrients to the bioreactor and to perform gaseous exchange. Any method for accomplishing gas exchange may be used, but in a preferred embodiment a length of tubing is used. The length of tubing may be wrapped around a stand, provided that sufficient amounts of the tubing are exposed to the atmosphere inside the incubator. Other types of gas exchangers that may be used include but are not limited to gas permeable Teflon bags. Description of an Embodiment of the Invention.

The bioreactor was built with 50 microcapillary fibers potted into a milled plastic assembly. The assembly had a 1-cm³ extra-capillary space, which was connected to two extra-capillary ports. These extra-capillary ports were present in the design in order to test the effect of the addition of reagents, such as hormones, growth factors or other biologically influential molecules, directly to the cells while the bioreactor is operating. Reagents introduced into these ports have the potential of remaining concentrated in the extra-capillary space and not being diluted into the circulating media depending on the microcapillary fiber molecular weight cutoff. The top and the bottom of the extra-capillary chamber were sealed with a #2 cover slip.

The assembly was connected in series to a medium reservoir bottle, a coiled up length of silicone tubing, and a pump fixture. The reservoir bottle had a capacity of 100-milliliters of medium, which was sufficient to sustain the 1-millimeter ovary fragment for a long period of time. The ovary fragment, or any other type of tissue, remains suspended among the bed of microcapillary fibers, which mimics the mass transfer of nutrients/wastes achieved by capillaries in the circulatory system. The long length of coiled silicone tubing was necessary to allow adequate time for gas exchange with the incubator's environment. Silicone tubing is specifically preferred because of the material's high permeability to physiological gases that are readily exchanged by the body to maintain homeostasis.

A pump fixture designed to fit a CellMax peristaltic pump or another commercially available peristaltic pump is needed to circulate medium around the closed circuit and through microcapillary fibers. A Cellmax Quad [Spectrum Laboratories Inc., Cat: CMQUAD-B] with the capability of simultaneously pumping four bioreactors was used.

Benefits of the Bioreactor.

The bioreactor of this invention is expected to allow scientist to do research at much reduced costs. Often research projects are dependent on observing time progressive phenomenon. This results in the need for hundreds of replicates in an experiment, with experimentation halting on a few replicates at a time in order to obtain time course data. For example, in a small experiment a hundred mice with liver tumors might be injected with a pharmaceutical drug and ten mice would be anesthetized and sampled each month for ten months. Biopsies of the liver tissues would reveal the time course effects of the pharmaceutical drug. Alternatively, liver tumor tissue could be placed in the bioreactor with the pharmaceutical drug and the histological changes could be photographed under a microscope as frequently as desired. Thus, there is a reduction in the number of animals being sacrificed and a reduction of general experimentation costs for initial screening.

This invention has potential societal benefits as well. Women that are scheduled to undergo chemotherapy or radiation treatments for cancer often have ovulated eggs removed, or part or all of their ovaries removed, and cryopreserved. This is done in order to provide eggs that can be fertilized and used to produce children should the women survive the cancer treatments. Radiation therapy and chemotherapy can cause mutations in the chromosomes of the egg, and therefore removal of the egg prior to cancer or other medical treatment circumvents this potential problem. In the past, ovarian tissue fragments have been cultured after cryopreservation (Hovatta, 1999), but such fragments had to be cultured in the kidney capsule (Oktay et al., 1998; Posada et al., 2001). The present invention would be a viable solution to enabling these women to have children.

Test Data And Analysis.

The tissue used to test the ability of the device to maintain tissue health was from the mouse ovary. For these experiments adult ovaries were removed from 7-week-old female mice and sliced into two thick segments. One segment from each ovary was place in the microcapillary bioreactor while the other segment from each ovary was placed in a standard cell culture dish and both preparations were covered with cell culture medium.

Two types of fibers were tested and are designated as Fiber A, which is made of cellulosic with a molecular weight cutoff 30 kD and Fiber B, which is made of high flux polysulfone with a molecular weight cutoff of 20 kD.

Ovary fragments grown with Fiber A appeared to spread along the fiber with increasing time of incubation. This spreading of cells along Fiber A may have occurred because the surface of Fiber A permitted cell attachment while the surface of Fiber B prevented cell attachment. In contrast fragments grown with Fiber B retained the original shape of the ovary fragment.

The ovary fragments grown with Fiber A appeared to produce mature follicles and subsequently eggs ovulated out of the follicles. The ability of the ovary fragments to produce mature follicles and to subsequently ovulate eggs indicated that the tissue had not died, and that the tissue retained its normal function (i.e., the production of follicles and eggs).

Ovary fragments in bioreactors made with Fiber A showed a darkening of some of the fibers over time when viewed under a microscope. The composition of this material was not determined. However since Fiber A allows adhesion of cells, the darkened portions of the fibers could represent sites where proteins from cells stuck to the fiber.

The ovary fragments in the bioreactors appeared healthy for the entire period of the experiment, that is 28 days. In contrast, visual observation of the ovarian fragments grown in the culture plate appeared to deteriorate over time.

Microcapillary Fiber Selection.

Microcapillary fibers are available in numerous lengths, diameters, materials, and molecular weight cutoffs. Fiber length is an important consideration for larger bioreactor designs, which are suitable for large aggregates of tissue. The bioreactors of the present invention may be built relatively small for viewing under high power microscopes and so readily available 10-centimeter length fibers may be utilized.

The material that the fibers are made from greatly affects the performance and function of the bioreactor. Depending on the surface properties of the material, use of a particular type of microcapillary fiber material will influence how the cells and tissue grow in the bioreactor. Some types of fibers will easily allow cell adhesion and encapsulations, and other types will prevent cell motility or scaffold incorporation. There are useful applications for particular fibers manufactured with material of any surface characteristic. Fiber material types conducive to cell motility could be used to increase an aggregate tissue mass by many folds whereas fiber material types prohibiting cell motility could be used as scaffolding to sustain an aggregate tissue for observations at increasing time intervals. The third fiber characteristic, molecular weight cutoff, has a highly significant influence on the function of the bioreactor. A proper fiber selection will allow easy diffusion of desired molecules to the cells while prohibiting molecules larger than the weight cutoff limit from reaching the cells. Table 1 lists molecules and their respective molecular weights for a comparison of molecular weight cutoff effects on a biological system.

Two types of fibers have been tested. These are nonlimiting examples of the brands and types of fibers which may be used. Fiber A is from a Cellmax Artificial Capillary module [Spectrum Laboratories Inc., Cat: 400-022], is made of cellulosic material and has a molecular weight cutoff 30 kD. The 30 kD cutoff allows for the diffusion of electrolytes in but does not permit large enzymes and factors to pass through much like the capillaries in a human circulatory system. The cellulosic is a substrate onto which cells can adhere and move around. This is in contrast to fiber B, which is from a FiberCell™ module [FiberCell™ Systems Inc., Cat: 4300-C2011], is made of high flux polysulfone, and has a molecular weight cut off of 20 kD. Fiber B's material has surface properties that are not conducive to cell adhesion nor cell motility and has a lower molecular weight cutoff that essentially reduces the quantity of biological components that are able to diffuse freely. However, FiberCell™ Systems Inc. Claims that fiber B's naturally hydrophilic polymer can improve cell viability by providing 10 times the gross filtration rate of cellulosic hollow fibers. Tables 2 and 3 below contain information about microcapillary fibers and their characteristic from FiberCell™ Systems Inc. and Spectrum Laboratories Inc.

TABLE 1

| | Molecular | Can diffuse | Can diffuse |
|---|---|---|---|
| Molecule Name | Weight | through Fiber A | through Fiber B |
| Water | 18 | ♦ | ♦ |
| Albumin | ˜68000 | | |
| Transferrin | 78000 | | |
| T3, Leu-4 (CD3γ) | 25000-28000 | ♦ | |
| T3, Leu-4 (CD3δ) | 20000 | ♦ | ♦ |
| T3, Leu-4 (CD3ε) | 20000 | ♦ | ♦ |
| T4, Leu-3 | 55000 | | |
| Neutral Endopeptidase | 100000 | | |
| Aminopeptidase | 150000 | | |
| Fas Antigen | 45000 | | |
| Fibrinogen (Protein) | ˜341000 | | |
| Urea (Waste) | 60.06 | ♦ | ♦ |
| Uric Acid (Waste) | 168.11 | ♦ | ♦ |
| Creatine (Waste) [C₄H₉N₃O₂] | 131.1 | ♦ | ♦ |
| Creatinine (Waste) [C₄H₇N₃O] | 113.12 | ♦ | ♦ |
| Bilirubin (Waste) [C₃₃H₃₆N₄O₆] | 584.68 | ♦ | ♦ |
| Glucose (Nutrients) [C₆H₁₂O₆] | 182.14 | ♦ | ♦ |
| Glycerol (Nutrients) [C₃H₈O₃] | 92.09 | ♦ | ♦ |
| Enzymes (Regulatory Substance) | >34000 | | |
| Oxygen (Gases) [O₂] | 32.00 | ♦ | ♦ |
| Carbon Dioxide (Gases) [CO₂] | 44.01 | ♦ | ♦ |
| Nitrogen (Gases) [N₂] | 28.01 | ♦ | ♦ |
| Na⁺ (Electrolytes) | 22.99 | ♦ | ♦ |
| K⁺ (Electrolytes) | 39.10 | ♦ | ♦ |
| Ca⁺ (Electrolytes) | 40.08 | ♦ | ♦ |
| Mg⁺ (Electrolytes) | 24.31 | ♦ | ♦ |
| Cl⁻ (Electrolytes) | 35.45 | ♦ | ♦ |
| HPO₄²⁻ (Electrolytes) | 95.97 | ♦ | ♦ |
| SO₄²⁻ (Electrolytes) | 96.06 | ♦ | ♦ |
| HCO₃⁻ (Electrolytes) | 61.01 | ♦ | ♦ |

Molecules, their molecular weight, and their ability to diffuse through fibers used in this experiment
(Fiber A = Cellmax module with 30 k MW cutoff, Fiber B = FiberCell module with 20 k MW cut off) (Abbas, 2000; Janeway, 1999)

TABLE 2
Microcapillary characteristics of cartridges available from
FiberCell Systems Inc.

| | | Surface | | | Packing | ECS | | MWCO | | Max. |
|---|---|---|---|---|---|---|---|---|---|---|
| Stock No. | Size | Area | | Fiber Type | Density | Vol | | 50% | | Cell# |
| 4300-C2025 | Small | 75 | cm2 | activated PS | 30% | 2.5 | mL | .2 | μm | 10⁸ |
| 4300-C2008 | Med. | 2100 | cm2 | low flux PS | 50% | 12 | mL | 5 | kd | 10⁹ |
| 4300-C2011 | Med. | 2100 | cm2 | high flux PS | 50% | 12 | mL | 20 | kd | 10⁹ |
| 4300-C2003 | Large | 1.2 | m2 | low flux PS | 50% | 70 | mL | 5 | kd | 5 × 10¹⁰ |
| 4300-C2018 | Large | 1.2 | m2 | high flux PS | 50% | 70 | mL | 20 | kd | 5 × 10¹⁰ |
| 4300-C2023 | Med. | 2100 | cm2 | cellulosic | 38% | 12 | mL | 10 | kd | 10⁹ |

TABLE 3
Microcapillary characteristics of cartridges available from
Spectrum Laboratories Inc.

| | | | Wall | Lumenal | Outer | | | | |
|---|---|---|---|---|---|---|---|---|---|
| Catalog | | O.D. | Thickness | Area | Surface | ECS | 95% | | 50% |
| Number | Material | μm | μm | cm² | Area cm² | Vol. | MWCO | | MWCO |
| 400-022 | Cellulosic | 210 | 15 | 220 | 250 | 2.2 | 150 | k | 30 k |
| 400-008 | Cellulosic | 216 | 8 | 780 | 847 | 7 | 20 | k | 4 k |
| 400-023 | Cellulosic | 200 | 10 | 1866 | 2100 | 12 | 50 | k | 10 k |
| 400-011 | Cellulosic | 210 | 15 | 1900 | 2200 | 12 | 150 | k | 30 k |
| 400-012 | Polyethylene | 430 | 50 | 964 | 1500 | 12 | 0.3 | μm | — |
| 400-014 | Polyethylene | 430 | 50 | 109 | 123 | 2.3 | 0.3 | μm | — |
| 420-015 | Polyethylene | 430 | 50 | 108 | 123 | 2.3 | 0.3 | μm | — |
| | coated | | | | | | | | |
| 400-007 | Polypropylene | 630 | 150 | 327 | 423 | 7 | 0.5 | μm | — |
| 420-007 | Polypropylene | 630 | 150 | 327 | 423 | 7 | 0.5 | μm | — |
| | coated | | | | | | | | |
| 400-025 | Polypropylene | 630 | 150 | 70 | 100 | 1.4 | 0.5 | μm | — |
| 420-025 | Polypropylene | 630 | 150 | 70 | 100 | 1.4 | 0.5 | μm | — |
| | coated | | | | | | | | |

Bioreactor Sterilization.

The bioreactors were gas sterilized with ethylene oxide, although other methods of sterilization may be utilized, provided that the structure and function of the bioreactor is not adversely affected. The sterilization procedure involved placing the bioreactor chambers inside gas permeable sterilization pouches and sealing the pouches with sterilization indicator tape. Inside each sterilization pouch were placed all other necessary components such as screws and luer lock caps. The complete pouches were then placed inside a gassing chamber. The chamber air was evacuated and ethylene oxide gas was introduced to replace the evacuated air. The bioreactors remained in contact with the ethylene oxide gas for 24-hours to insure that the sterilizing gas adequately diffused through all microcapillary tubes. This was followed by a 24-hour period of degassing where the ethylene oxide gas was evacuated and sterile air was continually introduced into the sterilization chamber. Prior to use the bioreactor's capillaries were given a final flush with sterile PBS.

Medium Preparation.

A laminar flow hood was prepared by spraying the inside with 70% ethanol while the hood was filtering the air through HEPA filters and by exposure to an ultraviolet germicidal lamp (253.7 nano-meter light) for 15 minutes. A 100-ml bottle of fetal bovine serum (FBS) [BioWhittaker, Cat: 14-502F] was thawed from freezer storage by using a warm water bath [Fisher Scientific, Model 220] at 37.0 degrees centigrade. After the 15-minute sterilization of the hood, the thawed FBS, a 1-liter bottle of Dulbecco's Modification of Eagle's Medium 1× (DMEM) [Mediatech Cellgro, Cat: 15-013-CM], and 10 steam autoclaved 100-ml bottles were placed in the hood aseptically. Following all aseptic techniques, the 100-ml of FBS was added to the 1 liter bottle of DMEM with 20,000 IU Pen-Strip antibiotics and 3 mM glutamate. The DMEM bottle cap was replaced snuggly and the bottle was inverted and reverted continually for approximately 1-minute to insure adequate mixing of the liquids. The bottle was gently inverted so that bubble formation was minimized. After this mixing, the prepared medium was aliquoted equally among the 10 steam autoclaved 100-ml bottles and these bottles were immediately placed in the refrigerator (8 degrees centigrade) for later use as needed.

Bioreactor Preparation & Ovary Fragment Inoculation.

To insure that all of the sterilizing ethylene oxide gas had been eliminated, a 100-ml bottle of Dulbecco's phosphate buffer solution 1× (PBS) [Mediatech Cellgro, Cat: 21-030-CM] was attached in place of the medium reservoir bottle and continuously circulated for four hours. This washing phase took place in a HEPA laminar flow hood [Ferma Scientific, Class IIA/B3 Biological Safety Cabinet] with the blower and the UV lamp operating. Meanwhile, four whole ovaries were removed from mice, cut into smaller gragments about one half the original size, and placed into petri dishes with the same medium prepared for the bioreactor. Next the PBS was completely pumped out of the system before the introduction of the DMEM containing 9% FCS. Sterile forceps were unpackaged and used to spread the microcapillary fibers apart so the ovary slices would be nestled in the bioreactor's microcapillary fibers. Each bioreactor was successively placed into the incubator as ovary fragment inoculation was completed. Upon the inoculation of all bioreactors, time lapsed photography began.

1. A microcapillary bioreactor comprising:
a body defining a chamber having an access opening;
a circulating medium passageway through the body and in communication with the chamber, the circulating medium passageway having an input port and an output port;
one or more semi-permeable microcapillary fibers disposed within the circulating medium passageway and traversing the chamber wherein each of the microcapillary fibers has an input end in fluid communication with the input port and an output end in fluid communication with the output port, the chamber and the one or more microcapillary fibers defining an intra-capillary space and an extra-capillary space within the chamber;
an extra-capillary input port in communication with the extra-capillary space;
and an extra-capillary output port in communication with the extra-capillary space.

2. The bioreactor according to claim 1 wherein the body includes access means for allowing placement of biological tissue into the chamber and removal of the biological tissue from the chamber.

3. The bioreactor according to claim 1 wherein the body includes a base and a cover that sealingly covers the chamber access opening and that exposes the chamber access opening when removed.

4. The bioreactor according to claim 1 wherein the body is generally plate-shaped with opposing front and rear faces.

5. The bioreactor according to claim 4 wherein at least one of the faces is generally flat.

6. The bioreactor according to claim 1 wherein the plate-shaped body has thickness that is within the focal length of a microscope.

7. The bioreactor according to claim 1 wherein the extra-capillary space has a volume of about one cm³.

8. The bioreactor according to claim 8 wherein the chamber is visible through a window in the body.

9. The bioreactor according to claim 1 wherein the chamber is visible through a window in the front face of the body and the chamber is also visible through a window in the rear face of the body.

10. The bioreactor according to claim 1 wherein the one or more microcapillary fibers has an exterior surface that permits cell attachment or encapsulation.

11. The bioreactor according to claim 1 wherein the one or more microcapillary fibers has an exterior surface that restricts cell attachment or encapsulation.

12. The bioreactor according to claim 1 wherein the one or more microcapillary fibers has an exterior surface that restricts cell motility.

13. The bioreactor according to claim 1 wherein the body is formed of polycarbonate.

14. The bioreactor according to claim 1 wherein the body is formed of acrylic.

15. The bioreactor according to claim 1 wherein at least a portion of the body is formed of a translucent material.

16. The bioreactor according to claim 1 wherein the microcapillary fibers have a molecular weight cutoff of about 30 kD.

17. The bioreactor according to claim 1 wherein the one or more microcapillary fibers have a molecular weight cutoff of about 20 kD.

18. The bioreactor according to claim 1 wherein the one or more microcapillary fibers have a molecular weight range of about 1 D to about 5000 kD.

19. The bioreactor according to claim 1 wherein the one or more microcapillary fibers are formed of a material selected from the group consisting of cellulosic, polyethylene, polypropylene and polysulfone.

20. The bioreactor according to claim 1 wherein the number of microcapillary fibers is selected based upon the size of the tissue explant being studied.

21. The bioreactor according to claim 1 wherein the one or more microcapillary fibers comprises about 50 individual fibers.

22. The bioreactor according to claim 1 wherein the one or more microcapillary fibers are included in a microcapillary assembly that is removably disposed within the circulating medium passageway.

23. A microcapillary bioreactor comprising:
a body defining a chamber having an access opening, the body having opposing generally flat front and rear faces, the chamber being visible through a window therein;
a circulating medium passageway through the body and in communication with the chamber, the circulating medium passageway having an input port and an output port;
one or more semi-permeable microcapillary fibers disposed within the circulating medium passageway and traversing the chamber wherein each of the one or more microcapillary fibers has an input end in fluid communication with the input port and an output end in fluid communication with the output port, the chamber and the microcapillary fibers defining an intra-capillary space and an extra-capillary space within the chamber;
an extra-capillary input port in communication with the extra-capillary space; and
an extra-capillary output port in communication with the extra-capillary space.

24. The microcapillary bioreactor of claim 23, wherein the microcapillary fibers are included in a microcapillary assembly that is removably disposed within the circulating medium passageway.

25. A microcapillary bioreactor system comprising:
a bioreactor according to claim 1;
a reservoir for holding a circulating medium, the reservoir being in fluid communication with the circulating medium input port; and
pump means for circulating the circulating medium from the reservoir, into the input port, through the microcapillary fibers and out the output port.

26. The bioreactor system of claim 25 wherein the pump means comprises a peristaltic pump.

27. The bioreactor system of claim 25 wherein the pump means circulates the circulating medium through gas-permeable tubing.

28. A method of evaluating biological tissue growth ex vivo, the method comprising:
providing a bioreactor according to claim 1;
innoculating the extra-capillary space of the chamber with the biological tissue;
circulating a suitable circulating medium through the microcapillary fibers;
incubating the bioreactor under conditions suitable to permit transfer of through the microcapillary fibers, and
evaluating the biological tissue in the extra-capillary space.

29. The method of evaluating biological tissue growth ex vivo of claim 28 further comprising the step of introducing a reagent into the bioreactor via the extra-capillary input port, and evaluating the effects of the reagent on the biological tissue.
